(19) United States
(12) Patent Application Publication
(10) Pub. No.: US 2003/0004409 A1
(43) Pub. Date: Jan. 2, 2003

(54) Magnetic resonance imaging apparatus with remotely controlled patient support

(75) Inventors: Mike Mueller, Nuremberg (DE); Dorothea Laux, Erlangen (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 09/896,676

(22) Filed: Jun. 29, 2001

(51) Int. Cl. A61B 5/05; A61B 5/055

(52) U.S. Cl. 600/410

(57) ABSTRACT

In a magnetic resonance imaging apparatus having a control console disposed at a location remote from the scanner which interacts with the examination subject, the control console has a display monitor at which a graphics display is presented having at least one control area thereon which, when activated, controls operation of a patient positioning mechanism for selectively positioning a patient thereon relative to an imaging volume in the scanner. The positioning of the examination subject and the overall control of an examination procedure are thereby controlled from a single location, with a precise visual indication of the position of the examination subject relative to the examination volume.

### BACKGROUND OF THE INVENTION

1. Field of the Invention

The present invention is directed to a magnetic resonance imaging apparatus of the type having a patient support which is operable to move a patient into and out of a scanner portion of the apparatus and to position the patient relative to the scanner portion of the apparatus.

2. Description of the Prior Art

A magnetic resonance imaging or tomography apparatus is a known type of imaging modality with which images of the interior of an examination subject can be obtained. Such an apparatus usually includes a scanner portion, which is the portion of the apparatus that interacts with the examination subject in order to obtain imaging data, and a processing and control portion which is disposed remote from the scanner portion but is in communication therewith via cables or some other communication link. The control and processing portion of the apparatus usually has an operator console, having a user interface, through which various parameters of the imaging process can be set and/or adjusted. Processing of the raw image data obtained from the scanning portion of the apparatus, to form a visible image, also takes place in the control and processing portion of the apparatus, as does display of the image.

The most commonly used type of scanner portion of a magnetic resonance imaging apparatus has a housing with a tubular opening (bore) proceeding longitudinally therethrough. Inside the housing and surrounding the bore are various sets of conductive coils which perform respectively different functions. One set of coils generates the basic, static magnetic field, and another set of coils, called gradient coils, generate gradient fields which are superimposed on the basic magnetic field for the purpose of spatially encoding the magnetic resonance signals which are obtained from examination subject. A radio-frequency coil can also be integrated in the housing, for emitting radio-frequency signals into the examination subject and for receiving the resulting nuclear magnetic resonance signals therefrom. If such a radio-frequency coil is integrated in the housing, it is usually a so-called whole-body coil. Additionally or alternatively, local coils can be employed which are physically placed relative to the examination subject to emit signals into, and receive signals from, localized portions of the examination subject.

Another type of configuration for the scanner portion of a magnetic resonance apparatus is a so-called “open” arrangement wherein permanent magnets are disposed spaced from and substantially parallel to each other in order to produce the basic magnetic field. Since continuous coils are not utilized for this purpose, this has the advantage of allowing side access to a patient in the imaging volume between the permanent magnets, so that surgical interventions can be undertaken without removing the patient from the imaging apparatus.

In either type of scanner configuration, it is necessary to move the patient into and out of the imaging volume, as well as to more precisely position the patient for the purpose of conducting a particular magnetic resonance examination. It may be necessary or desirable, for example, to successively move the patient incrementally through the imaging volume to obtain a series of images, such as of the spinal column.

For this purpose, mechanized, motor-operated patient support systems are known. Such systems can be designed as stand-alone systems, having a pedestal positioned next to the scanner housing, with a patient support plate being mounted on the pedestal so as to be movable into and out of the imaging volume. Alternatively, the support arrangement can be integrated with the housing of the scanner itself. In all instances, however, the control elements (user interface) for operating the patient support mechanism are located at the patient support mechanism itself. If adjustments in the position of the examination subject must be made during the course of an examination, and if only one person is available to operate the apparatus, this means that this person must move back and forth to operate the controls at the patient support mechanism and to operate the controls at the control console. If two persons are available to supervise the examination, one at the console and one at the controls of the patient support mechanism, this means that a communication arrangement, such as an intercom system, must be employed so that the person operating the patient support mechanism controls can be instructed by the person at the operating console as to when and how the position of the examination subject must be adjusted. Moreover, since the person operating the patient support mechanism controls is not able to see the display at the control console, this person must rely solely on the verbal descriptions and instructions relating to the necessary position adjustment. Such instructions and descriptions might be imprecise or incorrect, thereby resulting in inexact positioning, or positioning errors.

Moreover, the scanner housing may also contain auxiliary components for patient comfort, such as an illumination system in the bore, a ventilation system for providing air conditioning within the bore, and a video and/or audio system for providing entertainment to the examination subject during the sometimes lengthy examination. Also, other components may be provided at the scanner housing for assisting the examination, such as a light beam localizer or marking system. All of these types of additional components are also conventionally operated at the scanner housing and/or at the patient support mechanism.

Due to the extremely strong magnetic fields which are generated during the course of an examination in a magnetic resonance imaging apparatus, any electrical components, such as the controls for operating the patient support mechanism, must be shielded so that they are not damaged by or influenced by these strong magnetic fields. Moreover, if a surgical intervention is to take place while the patient is in the imaging apparatus, the controls must be sterilized along with the remainder of the apparatus.

### SUMMARY OF THE INVENTION

It is an object of the present invention to provide a magnetic resonance imaging apparatus which allows adjustment of the position of an examination subject relative to the imaging volume in a manner which minimizes errors and imprecise adjustments.

It is a further object of the present invention to provide such a magnetic resonance imaging apparatus wherein control of a magnetic resonance imaging examination, including adjustment of the position of the examination subject relative to the imaging volume, can be easily accomplished by one operator.

These objects are achieved in accordance with the principles of the present invention in a magnetic resonance imaging apparatus having a scanner which interacts with an examination subject to obtain image data using a magnetic resonance imaging sequence, and having a patient support mechanism adapted to position an examination subject relative to an imaging volume in the scanner, and wherein a user interface is provided at a location remote from the scanner and remote from the patient support mechanism which allows both the magnetic resonance imaging sequence and the positioning of the examination subject relative to the imaging volume to be controlled at a single location with the user interface.

The user interface can include a graphic display which is presented at the host computer which is used by an operator for supervising the overall examination. The display can include appropriate pictorial representations of the magnetic resonance scanner and the patient support, with appropriate icons for adjusting the patient support relative to the imaging volume, including the speed of movement of the movable patient plate on which the subject rests. The icons can be clicked on by an arrow movable by a mouse, or can be presented on a touch screen, or activated by a light pen.

The icons can include one or more icons which when clicked on or otherwise activated cause the patient support mechanism to execute a predetermined patient movement or bring about a predetermined result, such as automatically moving the subject on the patient plate to a predetermined fixed position, or a center position relative to the imaging volume or a position completely outside of the scanner bore, or to immediately interrupt (brake) an ongoing movement.

When operated by a mouse, the two-step procedure for activating one of these icons (i.e., positioning the cursor on the icon and depressing a key on the mouse) provides a level of safety so that these icons will not be inadvertently activated.

In addition to allowing a single operator to position the examination subject and control the examination from one location, which avoids errors and imprecise instructions as may occur when communication between two separated operators is necessary, the graphics display of the invention affords the additional advantage of providing the operator with a relatively precise representation of the position of the patient in the imaging volume. When a patient support mechanism operator is required in a conventional imaging apparatus having a tunnel or bore through which the examination subject must be moved, the patient support mechanism operator must necessarily look into the bore in order to see where the patient is positioned, and thus has only an end view, or a perspective view, of the patient's position. Providing a graphically displayed view allows the operator to more precisely see where the patient is located within the bore. Even in an open imaging apparatus, wherein the visual impediments caused by a closed tunnel are not present, it is still cumbersome for the patient support mechanism operator to obtain visual confirmation as to the exact position of the examination subject within the imaging volume.

### DESCRIPTION OF THE DRAWINGS

FIG. 1 is a schematic block diagram showing the basic components of a magnetic resonance imaging apparatus constructed and operating in accordance with the principles of the present invention.

FIG. 2 is a side view of an embodiment of the scanner and patient support mechanism which are controlled in accordance with the invention.

FIG. 3 illustrates an example of a graphics display for controlling the patient support mechanism in a magnetic resonance imaging apparatus in accordance with the invention.

### DESCRIPTION OF THE PREFERRED EMBODIMENTS

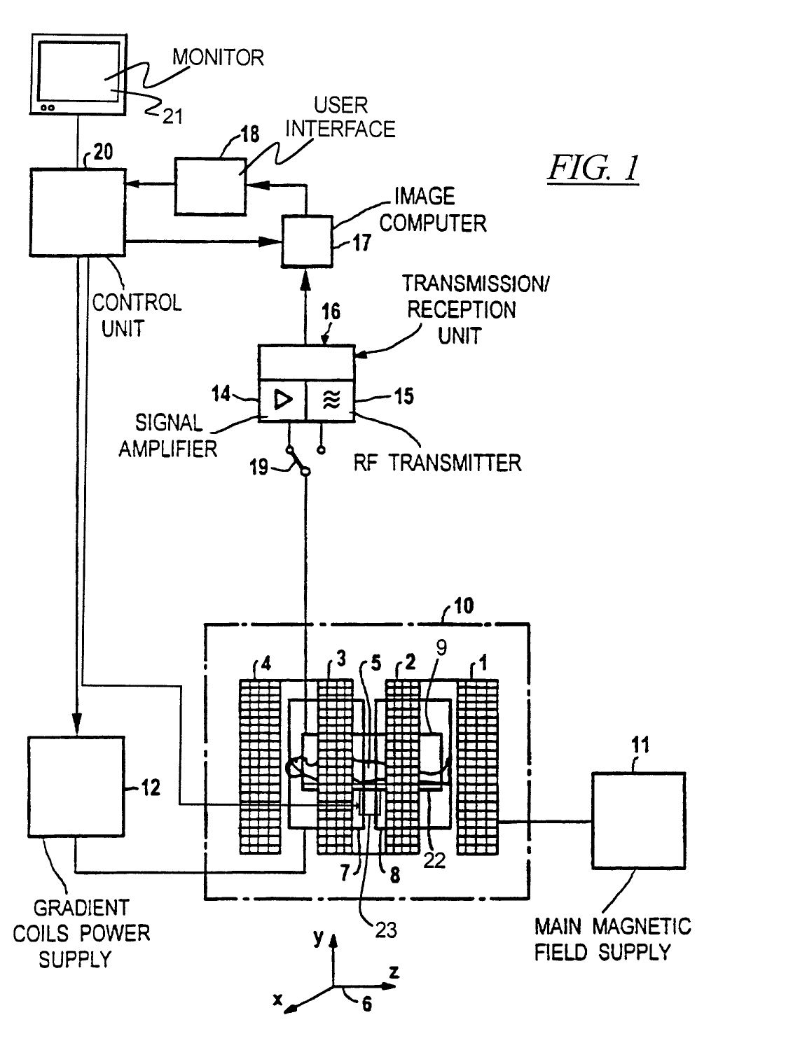

The magnetic resonance imaging system shown in FIG. 1 has a basic field magnet system formed by coils 1, 2, 3 and 4, which are supplied with current by a main magnetic field supply 11, so as to generate a static, homogenous basic magnetic field in an imaging volume into which a patient 5 can be moved. Gradient coils are also provided which respectively generate independent orthogonal magnetic field components in the x, y and z directions defined according to the Cartesian coordinate axes 6. For clarity, FIG. 1 shows only the gradient coils 7 and 8 which, together with a pair of identical gradient coils opposite thereto, generate the x-gradient. Identical y-gradient coils (not shown) lie parallel to, and above and below, the patient 5. Coils (not shown) for producing the z-gradient field are respectively disposed at the head and feet of the patient 5, oriented transversely to the longitudinal axis of the patient 5. The gradient coils are supplied with current from a gradient coils power supply 12.

As is known, the imaging apparatus also includes a radio-frequency antenna 9 which serves the purpose of triggering and receiving nuclear magnetic resonance signals in the patient 5. A transmission/reception unit 16 is connected to the radio-frequency antenna 9 via a switch 19. In a transmission mode, the switch 19 is operated to connect the radio-frequency antenna 9 to an RF transmitter 15, which causes radio-frequency signals to be emitted into the patient 5, thereby triggering nuclear magnetic resonance signals therein, at least in the imaging volume. In a reception mode, the switch 19 connects the radio-frequency antenna 9 to a signal amplifier 14, so that the arising nuclear magnetic resonance signals are supplied to an image computer 17. The image computer 17, in a known manner, generates an image of the examination subject therefrom, which is displayed on a monitor 21.

The overall examination sequence is controlled by a control unit 20 under a supervision of an operator via a user interface 18. The control unit 20 controls both the image computer 17 and the gradient coils power supply 12 to effect a selected pulse sequence for obtaining the magnetic resonance signals.

As also shown in FIG. 1, the control unit 20, via commands entered through the user interface 18, controls a schematically-indicated positioning unit 23, which is mechanically connected to a patient bed 22, on which the examination subject 5 lies during the examination.

The coils 1 through 4 of the basic magnet system as well as the gradient coils and the radio-frequency antenna 9 are all included in a scanner housing 10, into which the patient is moved by the positioning mechanism 23 and the patient bed 22 for the purpose of conducting the examination.

At least the user interface 18, the control unit 20 and the monitor 21 are disposed at a location remote from the scanner housing 10, which is necessary due to the extremely strong magnetic fields which are generated during the course of an examination, which can damage the control components. Therefore, typically there will be suitable shielding (not shown) between the room in which the scanner housing 10 is located and the control room. Usually there will be a window so that a person operating the user interface 18 has visual access to the scanner housing 10 and the patient 5.

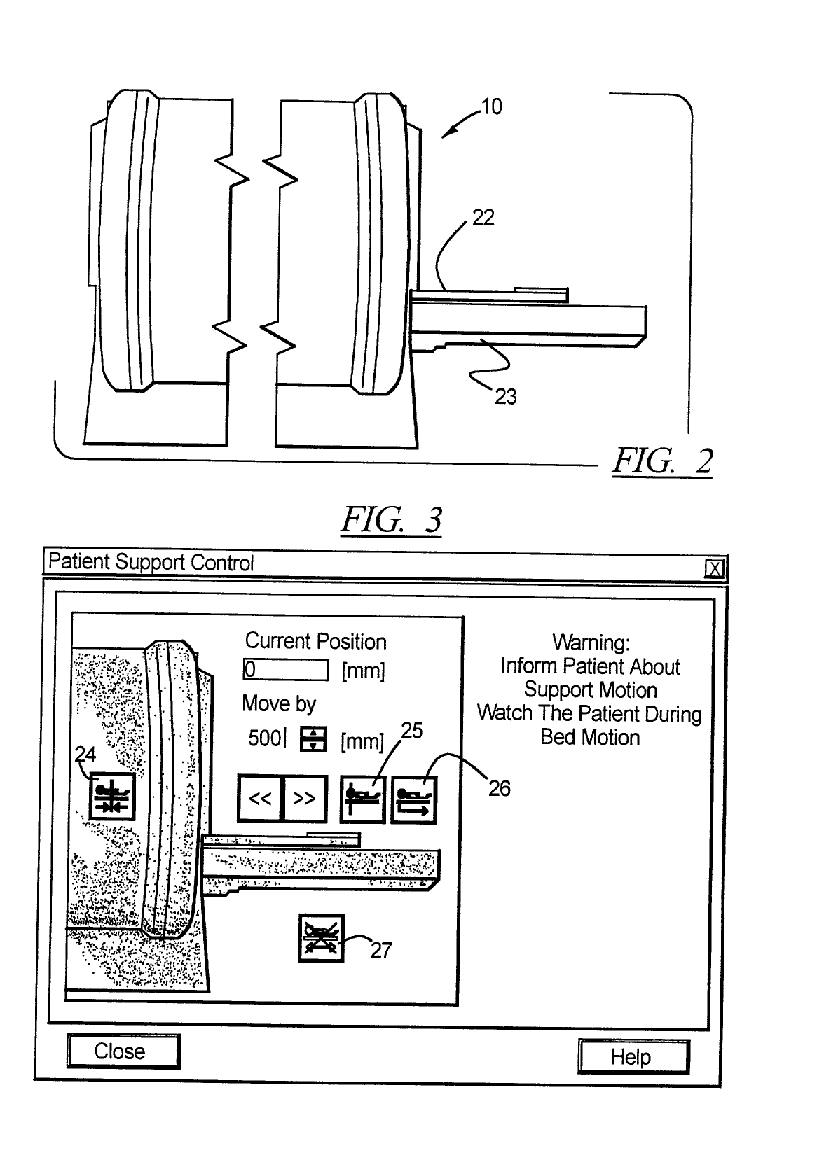

A typical configuration for the scanner housing 10 is shown in FIG. 2, with the patient bed 22 and the positioning mechanism 23 being integrated therewith. The scanner housing 10 has a cylindrical bore (tunnel) proceeding therethrough, into which and out of which the patient bed 22 is moved by the positioning mechanism 23.

An example of a graphics display which can be presented at the monitor 21 in accordance with the invention is shown in FIG. 3. In this example, a side view of the scanner housing 10 and the patient bed 22 and the positioning mechanism 23 is graphically illustrated, however, the view can be selected to coincide with the view that the operator has through the viewing window into the examination room, to provide appropriate visual correlation with the actual view seen by the operator, and the graphically represented view on the screen.

Via the user interface 18, the operator activates various control areas on the screen to operate the positioning mechanism 23 to selectively position the patient 5 relative to the imaging volume. The user interface 18 will include a keyboard and a mouse, but the control areas on the graphics display shown in FIG. 3 can alternatively be activated by a light pen, or the monitor 21 can include a touch screen on which the display shown in FIG. 3 is presented. The monitor 21 can be the same monitor as is used for displaying the examination control setting and/or the magnetic resonance image, or side-by-side monitors can be used to respectively display the graphics and the magnetic resonance image.

The exemplary display shown in FIG. 3 can be presented in the framework, for example, of an overall operating program for the magnetic resonance imaging apparatus, such as MREase which is a commercial operating program available from Siemens AG. In the embodiment shown in FIG. 3, a display region is provided which indicates, in millimeters, the current position of the patient bed 22, which can be precisely known and indicated since the starting position thereof is precisely known. Activating the control area designated “<<” indicates that movement of the patient bed 22 into or toward the imaging volume is to occur, activation of the control area designated “>>” indicates that movement out of or away from the imaging volume will occur. The amount of the movement is determined by the “Move by” portion of the display, which includes a numerical indication that can be entered via a keyboard as part of the user interface 18, or by activating the control areas with the up and down arrows which cause the displayed number to increase or decrease from the displayed number.

One or more further control areas in the form of icons, can be provided which, when activated, cause predetermined movements of the patient bed 22. Particularly in the context of mouse control, these icons can require a two-step procedure in order to be activated, thereby providing a measure of safety against inadvertent activation. First, the cursor must be moved to a position coinciding with the icon, and this must be followed by a mouse click on the icon.

These further icons can include, for example, an icon 24 which, when activated, causes the patient bed 22 to move to a predetermined, fixed position, which can be previously entered via the user interface 18, an icon 25 which, when activated, causes the patient bed 22 to move completely out of the scanner housing 10 to an extreme end position, an icon 25 which, when activated, automatically centers the patient bed 22 in the imaging volume, and an icon 26 which, when activated, automatically stops any movement of the patient bed 22.

In accordance with the invention, no controls at all must be provided at the positioning mechanism 23 itself, thereby avoiding the necessity of elaborate and expensive shielding for those controls, as well as avoiding a need for sterilization of those controls.

Other activation areas can be provided in the display shown in FIG. 3 for operating auxiliary components, such as tunnel lighting, ventilation and/or an entertainment system for occupying the patient during the examination, which sometimes can be relatively lengthy. Auxiliary components related to the examination itself also can have activation areas provided on the display shown in FIG. 3, such as a light beam marker.

The graphics display of the scanner housing 10 can be provided with additional details as needed, such as markers indicating the boundary of the tunnel, the positions of various coils, such as local coils, and other components. This allows the diagnostician to preserve, by making a screen shot, the exact location of all components of the apparatus at any given time.

Although modifications and changes may be suggested by those skilled in the art, it is the intention of the inventors to embody within the patent warranted hereon all changes and modifications as reasonably and properly come within the scope of their contribution to the art.

We claim as our invention:

1. A magnetic resonance imaging apparatus comprising:
a scanner housing having an opening and an imaging volume within said opening, said scanner housing containing components for producing and obtaining magnetic resonance signals in said imaging volume;
a support mechanism having a movable element adapted to receive an examination subject thereon for moving said examination subject into and out of said opening relative to said imaging volume;
a control unit, operated by a user interface, disposed remote from said scanner housing and said support mechanism and being in communication with said scanner housing and said support mechanism to operate said components in said scanner housing to obtain magnetic resonance signals from at least a portion of said examination subject in said imaging volume and to control said movable element to selectively position said examination subject relative to said imaging volume;
an image computer connected to said control computer and supplied with said magnetic resonance signals for producing image data therefrom; and
a display monitor arrangement connected to said control unit at which a visual image corresponding to said image data is displayed, and at which a graphic is also displayed providing a visual representation of said scanner housing and a position of said movable element relative to said imaging volume, said graphics display having at least one control area activatable by a user input entered via said user interface for controlling positioning of said movable element.

2. A magnetic resonance imaging apparatus as claimed in claim 1 wherein the representation of said scanner housing and position of said movable element in said graphics is selectable via said user interface.

3. A magnetic resonance imaging apparatus as claimed in claim 1 wherein said graphics include an identification of a current position of said movable element relative to said imaging volume.

4. A magnetic resonance imaging apparatus as claimed in claim 1 wherein said graphics include a first control area which, when activated via said user interface, causes movement of said movable element toward said imaging volume and a second control area which, when activated via said user interface, causes movement of said movable element away from said imaging volume.

5. A magnetic resonance imaging apparatus as claimed in claim 1 wherein said graphics include a control area which, when activated via said user interface, automatically moves said movable element to a center of said imaging volume.

6. A magnetic resonance imaging apparatus as claimed in claim 1 wherein said graphics include a control area which, when activated via said user interface, automatically moves said movable element to an extreme position out of said imaging volume.

7. A magnetic resonance imaging apparatus as claimed in claim 1 wherein said graphics include a control area which, when activated via said user interface, automatically moves said movable element to a predetermined fixed position relative to said imaging volume.

8. A magnetic resonance imaging apparatus as claimed in claim 1 wherein said graphics include a control area which, when activated via said user interface, immediately stops movement of said movable element.

9. A magnetic resonance imaging apparatus as claimed in claim 1 wherein said user interface includes a mouse for activating said at least one control area.

10. A magnetic resonance imaging apparatus as claimed in claim 1 wherein said opening in said scanner housing is a bore proceeding therethrough in which said imaging volume is disposed.
